(19) United States
(12) Patent Application Publication
(10) Pub. No.: US 2019/0150839 A1
(43) Pub. Date: May 23, 2019

(54) MATERIAL CHARACTERISTIC SIGNAL DETECTION METHOD AND APPARATUS

(71) Applicant: Analog Devices, Inc., Norwood, MA (US)

(72) Inventors: Neal Tait KURFISS, Somerville, MA (US); Shrenik DELIWALA, Andover, MA (US)

(73) Assignee: Analog Devices, Inc., Norwood, MA (US)

(21) Appl. No.: 15/816,609

(22) Filed: Nov. 17, 2017

(51) Int. Cl. A61B 5/00; A61B 5/1455

(52) U.S. Cl. CPC A61B 5/6843; H05B 33/0842; A61B 5/6826; A61B 5/14552

(57) ABSTRACT

Device for detecting characteristic signals of tissues and materials and methods for calculating thereof. Apparatus and methods for detecting the presence of tissue (skin) in pulse oximeter are disclosed. Specifically, analysis using optical techniques are used to identify characteristic signals of different compositions of matter. The present application discloses using a plurality of photodetectors at different distances from a source to measure light scattering through a variety of materials, and more specifically, to detect the presence of tissue.

### FIELD OF THE DISCLOSURE

The present disclosure relates to delineating material using characteristic signatures, primarily in pulse oximetry. More specifically, this disclosure describes apparatus and techniques relating to optically identifying substances and changing power modes as a result thereof.

### BACKGROUND

Oximeters are photoelectric devices which measure the oxygen saturation of blood. Historically, these devices were first used in clinical laboratories on samples of blood taken from patients. In recent years, non-invasive oximeters have been developed and are now widely used in intensive care units to monitor critically ill patients and in operating rooms to monitor patients under anesthesia. Early non-invasive devices relied on dialization of the vascular bed in, for example, the patient's ear lobe to obtain a pool of arterial blood upon which to perform the saturation measurement.

More recently, non-invasive devices known as “pulse oximeters” have been developed which rely on the patient's pulse to produce a changing amount of arterial blood in, for example, the patient's finger or other selected extremity. Pulse oximeters for home use are small, lightweight monitors that painlessly attach to a fingertip to monitor the amount of oxygen carried in the body. An oxygen level of greater than 95% is generally considered to be a normal oxygen level. An oxygen level of 92% or less (at sea level) suggests a low blood oxygen.

Pulse oximetry is a noninvasive method for monitoring a person's oxygen saturation (SO₂). Though its reading of SpO₂ (peripheral oxygen saturation) is not always identical to the more desirable reading of SaO₂ (arterial oxygen saturation) from arterial blood gas analysis, the two are correlated well enough that the safe, convenient, noninvasive, inexpensive pulse oximetry method is valuable for measuring oxygen saturation in clinical use.

Pulse oximeters measure oxygen saturation by (1) passing light of two more selected wavelengths, e.g., a “red” wavelength and an “IR” wavelength, through the patient's extremity, (2) detecting the time-varying light intensity transmitted through the extremity for each of the wavelengths, and (3) calculating oxygen saturation values for the patient's blood using the: Lambert-Beers transmittance law and the detected transmitted light intensities at the selected wavelengths.

Less commonly, reflectance pulse oximetry is used as an alternative to transmissive pulse oximetry described above. This method does not require a thin section of the person's body and is therefore well suited to a universal application such as the feet, forehead, and chest, but it also has some limitations. Vasodilation and pooling of venous blood in the head due to compromised venous return to the heart can cause a combination of arterial and venous pulsations in the forehead region and lead to spurious SpO₂ results. Such conditions occur while undergoing anesthesia with endotracheal intubation and mechanical ventilation or in patients in the Trendelenburg position.

For people with COPD, asthma, Congestive Heart Failure (CHF) and other conditions, pulse oximetry is a technology used to measure the oxygen level in your blood and your heart rate. Using a clip to a patient's fingertip, a finger pulse oximeter is equipped with technology to detect changes in your blood oxygen level.

In its most common (transmissive) application mode, a sensor device is placed on a thin part of the patient's body, usually a fingertip or earlobe, or in the case of an infant, across a foot. The device passes two wavelengths of light through the body part to a photodetector. It measures the changing absorbance at each of the wavelengths, allowing it to determine the absorbances due to the pulsing arterial blood alone, excluding venous blood, skin, bone, muscle, fat, and (in most cases) nail polish.

Personal fingertip oximeters are small, portable devices which are frequently carried with the patient. However, the inventors of the present disclosure have recognized shortcomings in the state of the art. Specifically, pulse oximeters exhibit a poor capacity for distinguishing compositions which are disposed within them. That is, pulse oximeters cannot determine whether tissue (e.g., a fingertip) is present within them or another material.

This overview is intended to provide an overview of subject matter of the present patent application. It is not intended to provide an exclusive or exhaustive explanation of the invention. Further limitations and disadvantages of conventional and traditional approaches will become apparent to one of skill in the art, through comparison of such systems with some aspects of the present invention as set forth in the remainder of the present application with reference to the drawings.

### SUMMARY OF THE DISCLOSURE

Device for detecting characteristic signals of tissues and materials and methods for calculating thereof. Apparatus and methods for detecting the presence of tissue (skin) in pulse oximeter are disclosed. Specifically, analysis using optical techniques are used to identify characteristic signals of different compositions of matter. The present application discloses using a plurality of photodetectors at different distances from a source to measure light scattering through a variety of materials, and more specifically, to detect the presence of tissue.

According to one aspect, the present disclosure is an apparatus for identifying a material using optical analysis techniques described herein. Specifically, the apparatus is disposed in close proximity to the surface of a material and detection and identification is executed thereto.

According to another aspect of the device, light is transmitted through the material through which it is scattered by the material.

According to another aspect, the scattered light is incident upon a plurality of detectors, each of which are disposed at various distance relative to a light source from the light was transmitted.

According to another aspect, the ratio(s) of detected light is used to determine the presence of tissue.

According to another aspect, the ratio(s) of detected light is used to determine and identify the material disposed in close proximity to the apparatus.

According to another aspect, the apparatus uses a look-up table disposed in non-volatile memory.

According to another aspect, the look-up table comprising ratios of known and/or predetermined materials.

According to yet another aspect, the apparatus utilizes logic which when executed performs the steps in receiving the light information and making a material determination.

According to another aspect, the present disclosure comprises an analog front-end in electrical communication with one or more photodetectors and/or pulse oximeters.

The drawings show exemplary pulse oximeter circuits and configurations. Variations of these circuits, for example, changing the positions of, adding, or removing certain elements from the circuits are not beyond the scope of the present invention. The illustrated pulse oximeters, configurations, and complementary devices are intended to be complementary to the support found in the detailed description.

### BRIEF DESCRIPTION OF THE DRAWINGS

For a fuller understanding of the nature and advantages of the present invention, reference is made to the following detailed description of preferred embodiments and in connection with the accompanying drawings, in which:

FIG. 1 shows an exemplary pulse oximeter device, in accordance with some embodiments of the disclosure provided herein;

FIG. 2 depicts the front view of an exemplary material characteristic signal detection device, in accordance with some embodiments of the disclosure provided herein;

FIG. 3 depicts a left-side view of an exemplary material characteristic signal detection device, in accordance with some embodiments of the disclosure provided herein;

FIG. 4 depicts a right-side view of an exemplary material characteristic signal detection device, in accordance with some embodiments of the disclosure provided herein;

FIG. 5 depicts a top-down view of an exemplary material characteristic signal detection device, in accordance with some embodiments of the disclosure provided herein;

FIG. 6 demonstrates an exemplary material characteristic signal detection device in operation, in accordance with some embodiments of the disclosure provided herein;

FIG. 7 graphically illustrates the calculated operation of an exemplary material characteristic signal detection device, in accordance with some embodiments of the disclosure provided herein; and,

FIG. 8 is an exemplary table demonstrating the calculated operation of an exemplary material characteristic signal detection device, in accordance with some embodiments of the disclosure provided herein.

### DETAILED DESCRIPTION

The present disclosure relates to delineating material using characteristic signatures, primarily in pulse oximeter. More specifically, this disclosure describes apparatus and techniques relating to optically identifying substances and changing power modes as a result thereof.

The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Other objects, advantages and novel features of the disclosure are set forth in the proceeding in view of the drawings where applicable.

Pulse oximetry is at present the standard of care for the continuous monitoring of arterial oxygen saturation (SpO₂). Pulse oximeters provide instantaneous in-vivo measurements of arterial oxygenation, and thereby provide early warning of arterial hypoxemia, for example.

A pulse oximeter comprises a computerized measuring unit and a probe attached to the patient, typically to his or her finger or ear lobe. The probe includes a light source for sending an optical signal through the tissue and a photo detector for receiving the signal after transmission through the tissue. On the basis of the transmitted and received signals, light absorption by the tissue can be determined.

During each cardiac cycle, light absorption by the tissue varies cyclically. During the diastolic phase, absorption is caused by venous blood, tissue, bone, and pigments, whereas during the systolic phase there is an increase in absorption, which is caused by the influx of arterial blood into the tissue. Pulse oximeters focus the measurement on this arterial blood portion by determining the difference between the peak absorption during the systolic phase and the constant absorption during the diastolic phase. Pulse oximetry is thus based on the assumption that the pulsatile component of the absorption is due to arterial blood only.

Light transmission through an ideal absorbing sample is determined by the known Lambert-Beer equation as follows:

Iₒᵤₜ=Iᵢₙe⁻^{_{ε}}^{DC}

where, Iᵢₙ is the light intensity entering the sample, Iₒᵤₜ, is the light intensity received from the sample, D is the path length through the sample, ε is the extinction coefficient of the analyte in the sample at a specific wavelength, and C is the concentration of the analyte. When Iᵢₙ, D, and E are known, and Iₒᵤₜ is measured, the concentration C can be calculated.

In pulse oximetry, in order to distinguish between the two species of hemoglobin, oxyhemoglobin (HbO₂), and deoxyhemoglobin (RHb), absorption must be measured at two different wavelengths, i.e. the probe includes two different light emitting diodes (LEDs). The wavelength values widely used are 660 nm (red) and 940 nm (infrared), since the said two species of hemoglobin have substantially different absorption values at these wavelengths. Each LED is illuminated in turn at a frequency which is typically several hundred Hz.

Pulse oximeters tend to be small, portable devices which fingertips are inserted thereto. The inventors of the present disclosure of recognized that when the device has trouble distinguishing when materials other than tissue are present in the orifice of the device. The can occur when the pulse oximeter is being carried within a bag and a pen, for example, is inadvertently positioned into the orifice.

The current state of the art intermittently pulses high intensity light in an attempt to detect a heart-rate signal. This tends to drain the device's battery very quickly. The inventors of the present disclosure have developed and device and method to more robustly determine the presence of tissue disposed in a pulse oximeter. In some embodiments, the device and method can optically analyze and identify other materials, as well, using low intensity light thereby preserving battery life.

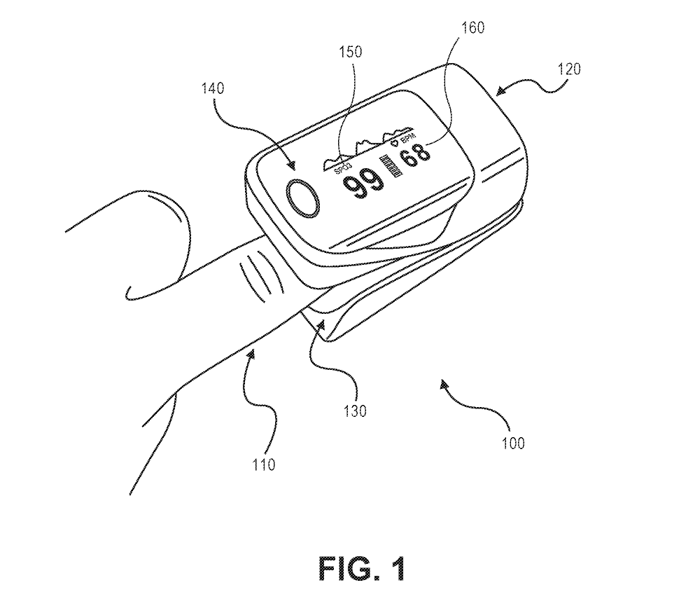

FIG. 1 shows an exemplary pulse oximeter device 100, in accordance with some embodiments of the disclosure provided herein. Pulse oximeter device 100 comprises body 120, function button 140, SpO₂ display 150, PR display 160, and finger orifice 130.

Body 120 is constructed in a spring-loaded clothespin fashion. It allows the orifice 130 to securely but safely clamp onto a patient's finger 110. Depending on embodiment/model, function button 140 can be used for on-off power, cycling through modes, cycling through displays and/or checking battery power levels, any of which are not beyond the scope of the current disclosure.

SpO₂ display 150 outputs current blood oxygen saturation level as a percentage (unitless). Blood oxygen saturation level (SpO₂) is a measure of the amount of oxygen carried in the hemoglobin. SpO₂ is expressed as a percentage of the maximum amount of oxygen that hemoglobin in the blood can carry. Since hemoglobin accounts for over 90% of oxygen in blood, SpO₂ also measures the amount of oxygen in blood. PR display 160 outputs current pulse rate in units of beats per minute (bpm).

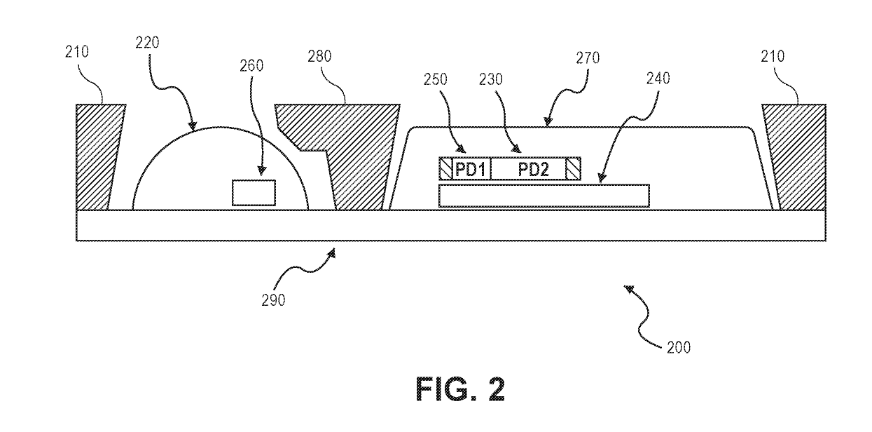

FIG. 2 depicts the front view of an exemplary material characteristic signal detection device 200, in accordance with some embodiments of the disclosure provided herein. Material characteristic signal detection device 200 comprises ambient light blocking members 210, opto-isolator 280, substrate 290, light emitting diode (LED) 260, LED cover 220, analog front end (AFE) 240, photodetector (PD₁) 250, photodetector (PD₂) 230, and photodetector cover 270.

In one or more embodiments, substrate 290 is a die manufactured from Silicon on Chip (SoC) fabrication processes which are known in the art, however any suitable support structure is not beyond the scope of the present disclosure. For example, substrate 290 can be made from any metal, semi-metallic, semiconductor, mixture/compound or polymer, provided caution is taken to ensure the AFE 240 is not shorted.

Ambient light blocking members 210 run along the perimeter of the upper substrate 290. Their function is to block ambient light from being received by photodetectors 230, 250. As such, ambient light blocking members 210 are made from an opaque polymer and/or lossy material having a thickness much greater than the average skin depth, according to some embodiments of the present invention. High conductivity (mirrored) are also not beyond the scope of the present disclosure.

Similarly, opto-isolator 280 traverses the entire span between the LED 260 side and the photodetectors 230, 250 side of the device, which will be explained in greater detail later in the disclosure. The function of the opto-isolator 280 is to block LED 260 light from being directly received by photodetectors 230, 250. As such, opto-isolator 280 are made from an opaque polymer and/or lossy material having a thickness much greater than the average skin depth, according to some embodiments of the present invention. High conductivity (mirrored) are also not beyond the scope of the present disclosure, however, this is not a preferred embodiment as will be clear later in the disclosure.

In one or more embodiments, LED 260 is an off-the-shelf green (495 nm-570 nm) light emitting diode. However, any suitable, compact light producing device is not beyond the scope of the present disclosure—whether coherent, incandescent, or even thermal black-body radiation, etc. LED cover 230 is a transparent polymer protective encasement of LED 260. In other embodiments, LED cover 230 is crystalline (glass, Pyrex, etc.), although any suitable can be used. While translucent and/or lossy materials may be used within the scope of the present disclosure, these are not preferred embodiments, which will be made more apparent later in the disclosure.

Photodetectors 250, 230 (PD₁, PD₂, respectively) are sensors of light or other electromagnetic energy. Photodetector 230, 250 have p-n junctions that converts light photons into current. The absorbed photons make electron-hole pairs in the depletion region, which is used to detect received light intensity. In some embodiments, photodetectors 250, 230 are photodiodes or phototransistors. However, any light detecting means, e.g., avalanche, photo-multiplier tube, etc. is not beyond the scope of the present disclosure.

Analog front-end 240 (AFE or analog front-end controller AFEC) is a set of analog signal conditioning circuitry that uses sensitive analog amplifiers, often operational amplifiers, filters, and sometimes application-specific integrated circuits for sensors, radio receivers, and other circuits to provide a configurable and flexible electronics functional block, needed to interface a variety of sensors to an, antenna, analog to digital converter or in some cases to a microcontroller. AFE 240 is in electrical communication with photodetectors 250, 230 and pulse oximeter 100.

Photodetector cover 270 is a transparent polymer protective encasement of PD₁ and PD₂. In other embodiments, photodetector cover 270 is crystalline (glass, Pyrex, etc.), although any suitable can be used. While translucent and/or lossy materials may be used within the scope of the present disclosure, these are not preferred embodiments, which will be made more apparent later in the disclosure.

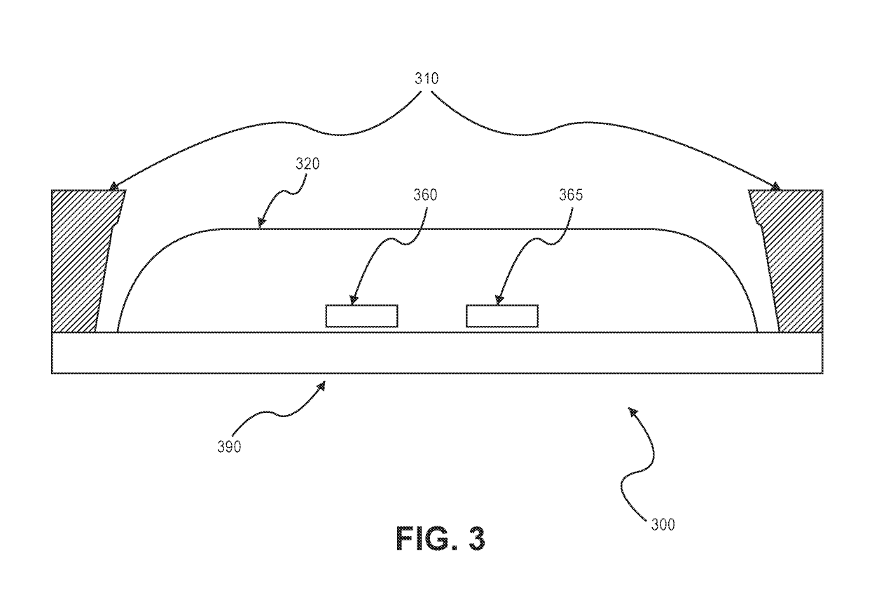

FIG. 3 depicts a left-side view of an exemplary material characteristic signal detection device 300, in accordance with some embodiments of the disclosure provided herein. Material characteristic signal detection device 300 comprises ambient light blocking members 310, substrate 390, light emitting diode (LED) 360, light emitting diode (LED) 365, and LED cover 320.

Ambient light blocking members 310 run along the perimeter of the upper substrate 390. Their function is to block ambient light from being received by photodetectors 230, 250. As such, ambient light blocking members 310 are made from an opaque polymer and/or lossy material having a thickness much greater than the average skin depth, according to some embodiments of the present invention. High conductivity (mirrored) are also not beyond the scope of the present disclosure.

In one or more embodiments, substrate 390 is a die manufactured from Silicon on Chip (SoC) fabrication processes which are known in the art, however any suitable support structure is not beyond the scope of the present disclosure. For example, substrate 390 can be made from any metal, semi-metallic, semiconductor, mixture/compound or polymer.

LED cover 320 is a transparent polymer protective encasement of LEDs 360, 365. In other embodiments, LED cover 320 is crystalline (glass, Pyrex, etc.), although any suitable can be used. In one or more embodiments, LEDs 360, 365 are an off-the-shelf green (495 nm-570 nm) light emitting diode. However, any suitable, compact light producing device is not beyond the scope of the present disclosure.

In other embodiments, light emitting diode (LED) 360 and light emitting diode (LED) 365 are different colors, which are not limited to the visible spectrum. The inventors of the present disclosure note that accuracy of material detection can be augmented by analysis using a plurality of wavelengths. While this will be discussed in greater detail later in the disclosure, a material's characteristic signal is based upon scattering and absorption which is wavelength dependent (dispersion, imaginary part of the its complex impedance, etc.), at least in part.

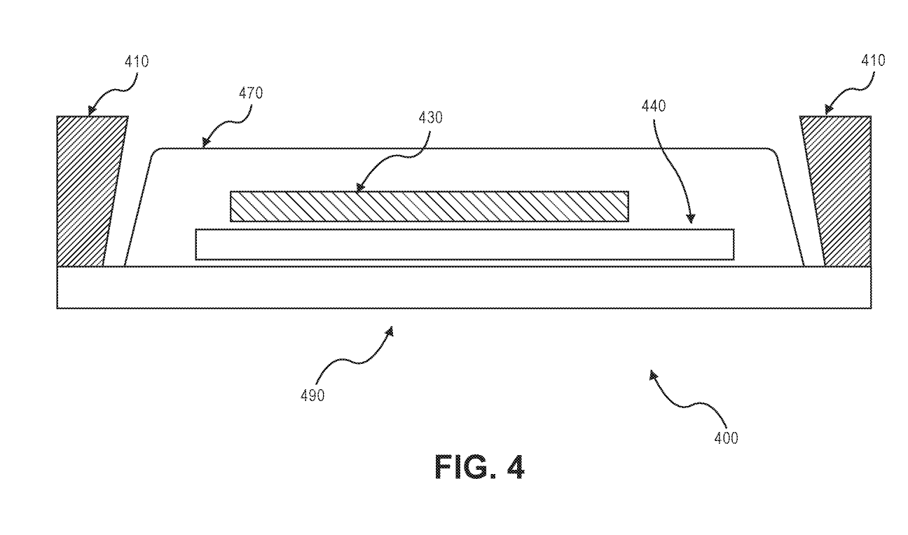

FIG. 4 depicts a right-side view of an exemplary material characteristic signal detection device 400, in accordance with some embodiments of the disclosure provided herein. Material characteristic signal detection device 400 comprises ambient light blocking members 410, substrate 490, analog front end (AFE) 440, photodetector (PD₂) 230, and photodetector cover 470.

As stated above, substrate 490 is a die manufactured from Silicon on Chip (SoC) fabrication processes which are known in the art, however any suitable support structure is not beyond the scope of the present disclosure. For example, substrate 490 can be made from any metal, semi-metallic, semiconductor, mixture/compound or polymer.

Similarly, as previously described, ambient light blocking members 410 run along the perimeter of the upper substrate 490. Their function is to block ambient light from being received by photodetector 430. As such, ambient light blocking members 410 are made from an opaque polymer and/or lossy material having a thickness much greater than the average skin depth, according to some embodiments of the present invention.

Photodetector cover 470 is a transparent polymer protective encasement of photodetector 430 et al. In other embodiments, photodetector cover 470 is crystalline (glass, Pyrex, etc.), although any suitable can be used.

Analog front-end 440 (AFE) is a set of analog signal conditioning circuitry that uses sensitive analog amplifiers, operational amplifiers, filters, and application-specific integrated circuits as needed to interface with sensors to analog to digital converter and/or microcontroller. AFE 440 is in electrical communication with photodetectors 230 and pulse oximeter 100.

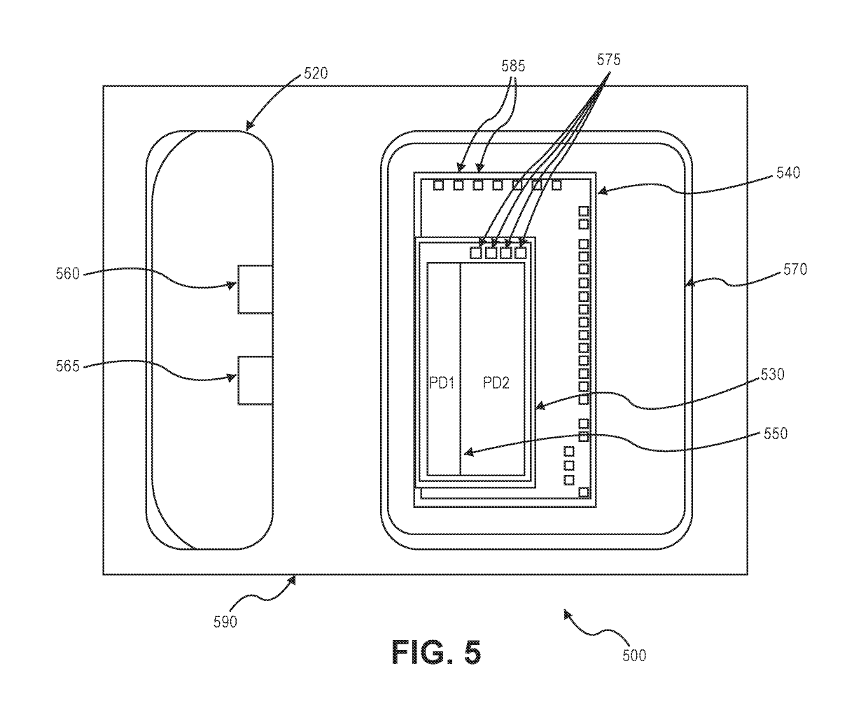

FIG. 5 depicts a top-down view of an exemplary material characteristic signal detection device 500, in accordance with some embodiments of the disclosure provided herein. Material characteristic signal detection device 500 comprises substrate 590, light emitting diodes (LED) 560, 565, LED cover 520, analog front end (AFE) 540, photodetector (PD₁) 550, photodetector (PD₂) 530, photodetector cover 570, PD pin-out 575, and AFE pin-out 585.

As stated above, substrate 590 is a die manufactured from Silicon on Chip (SoC) fabrication processes which are known in the art, however any suitable support structure is not beyond the scope of the present disclosure.

Similarly, as previously described, photodetector cover 570 and LED cover 520 are transparent polymer protective encasements of photodetectors 530, 550 and LEDs 560, 565, respectively. In other embodiments, photodetector cover 570 and LED covers are crystalline (glass, Pyrex, etc.), although any suitable can be used.

In one or more embodiments, LEDs 560, 565 are an off-the-shelf green (495 nm-570 nm) light emitting diode. However, any suitable, compact light producing device of any color is not beyond the scope of the present disclosure. In some embodiments where LEDs, 560, 565 emit different wavelengths, photodetector (PD₁) 550 and photodetector (PD₂) 530 can be modified to accommodate the detection thereof. For example, half of photodetector (PD₁) 550 and photodetector (PD₂) 530 can be covered with different optical filters.

In particular, photodetector (PD₁) 550 and photodetector (PD₂) 530 can be covered with dichroic filters, at least in part. A dichroic filter, thin-film filter, or interference filter is a very accurate color filter used to selectively pass light of a small range of colors while reflecting other colors. By comparison, dichroic mirrors and dichroic reflectors tend to be characterized by the color(s) of light that they reflect, rather than the color(s) they pass.

While dichroic filters are used in the present embodiment, other optical filters are not beyond the scope of the present invention, such as, interference, absorption, diffraction, grating, Fabry-Perot, etc. An interference filter consists of multiple thin layers of dielectric material having different refractive indices. There also may be metallic layers. In its broadest meaning, interference filters comprise also etalons that could be implemented as tunable interference filters. Interference filters are wavelength-selective by virtue of the interference effects that take place between the incident and reflected waves at the thin-film boundaries.

In other embodiments, a plurality of detectors is implemented, e.g., at least two for wavelength such that each of the pair of the plurality is wavelength specific. For example, there are at least two detectors (PD₁, PD₂) for every light emitting diode for a particular lambda.

Analog front-end 540 (AFE) is a set of analog signal conditioning circuitry that uses sensitive analog amplifiers, operational amplifiers, filters, and application-specific integrated circuits as needed to interface with sensors to analog to digital converter and/or microcontroller.

AFE 540 is in electrical communication with photodetectors 530, 550 through PD pin-outs 575. PD pin-outs 575 electrically communicate with photodetectors 530, 550 through traces. In the present embodiment, photodetectors 530, 550 and AFE 540 are packed as dies with soldered pin-outs. However, in other embodiments, they are integrated at the wafer level communicating through traces and vertical interconnect access (VIA) or through silicon VIA (TSV).

In some embodiments, AFE pin-out 585 is electrical communication with pulse oximeter 100. In other embodiments, AFE pin-out 585 can be in electrical communication with a microcontroller unit (MCU), field programmable gate array (FPGA), bus, or other computer platform, such as, Arduino or Raspberry Pi, etc.—all of which are not beyond the scope of the present disclosure.

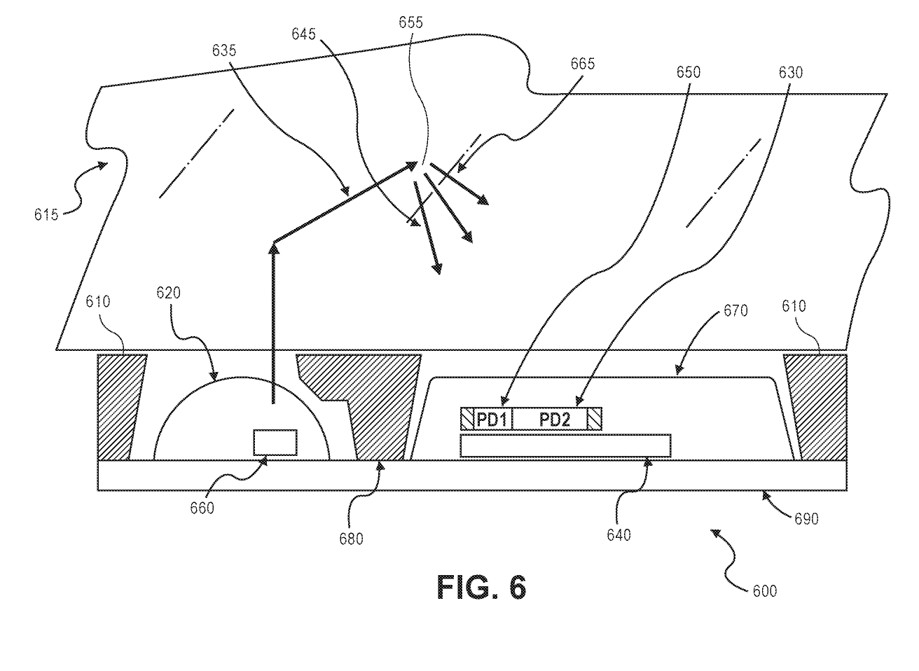

FIG. 6 demonstrates an exemplary material characteristic signal detection device 600 in operation, in accordance with some embodiments of the disclosure provided herein. Material characteristic signal detection device 600 comprises ambient light blocking members 610, opto-isolator 680, substrate 690, light emitting diode (LED) 660, LED cover 620, analog front end (AFE) 640, photodetector (PD₁) 650, photodetector (PD₂) 630, and photodetector cover 670.

In operation, material characteristic signal detection device 600 is disposed next to a patient's skin (i.e., tissue 615). Material characteristic signal detection device 600 egresses light from LED 660 and LED cover 620 which is incident upon tissue 615. In addition to blocking direct lighting into photodetector 650, opto-isolator 680 serves restrict transmission of light at an angle greater than orthogonal relative to the plane of LED 660. Lensing can also be used to substantially restrict the light to substantially orthogonal to the plane, in other embodiments.

Referring to FIG. 6, some of the light propagating through tissue 615 is absorbed while some of it scattered—predominately Mie with Rayleigh having a much smaller contribution. Both absorption and scattering are parameters which can be used to identify the presence of tissue 615. Yet, for the purpose of the present figure description, scattering will now be explained, albeit in somewhat heuristic ray tracing terms.

Scattered ray 635 gets decomposed by scattering within tissue 615. Those in the art will recognize that the ray decomposition is an oversimplification. However, the vector magnitudes of scatter rays 645, 655 and 665 are useful in demonstrating relative intensity absorption as a function of distance from LED 660. That is, scattered ray 645 has a greater intensity when incident upon photodetector 650. While scattered ray 655 has smaller magnitude (intensity) when incident upon photodetector 630. This is a function of propagation distance within tissue 615. More specifically, the further light travels through tissue 615, the more it is subject to scattering and absorption thereby resulting in decreased intensity. The significance of which will now be described in greater detail.

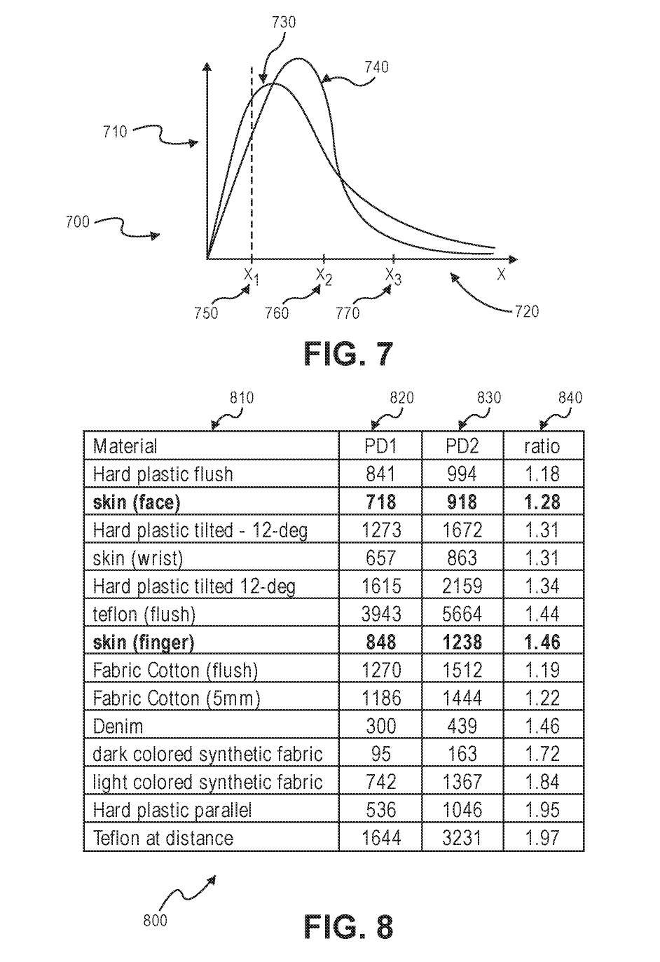

FIG. 7 graphically illustrates the calculated operation of an exemplary material characteristic signal detection device, in accordance with some embodiments of the disclosure provided herein. The inventors of the present disclosure have observed in reference to FIG. 7 is based upon an expectation of the scattering theory of light. That is, different materials with scattering and absorption characteristics generate different spatial distributions of light output with respect to the light input.

Chart 700 attempts to illustrate what the inventors of the present disclosure have discovered, at least in part. In the present embodiment, Y-axis 710 is intensity, I. However, Y-axis 710 can be energy flux (Poynting vector), incident power or photodetector voltage/current. X-axis 720 represents distance from the source, for example, along the plane of the substrate 690 with respect to the discussion of the recent embodiment.

As a function of distance, the inventors of the present disclosure have observed that light intensity substantially follow a curve depicted in FIG. 7. Intensity curves 730, 740 represent different materials. These characteristic curves for different material are measured and exploited. It is noted that while analytical intensity curves 730, 740 are representations of intensities as a function of distance, the region between the origin (x=0) and x₁ 750 is not physically accurate. Meaning x₁ 750 is a boundary condition and anything less than x₁ 750 (close to the source) is ignored.

Referring to chart 700 of FIG. 7 in reference to FIG. 6, let x₂ 760 be the mean distance of photodetector 650 (PD₁) from the source, i.e., LED 660. Let x₃ 770 be the mean distance of photodetector 630 (PD₂) from the source, i.e., LED 660. Graphically, it can be seen in FIG. 7 that intensity curves 730, 740 exhibit different values at both x₂ 760 and x₃ 770. These values are used to identify a material disposed next to an exemplary material characteristic signal detection device which will now be discussed in greater detail.

FIG. 8 is an exemplary table 800 demonstrating the calculated operation of an exemplary material characteristic signal detection device, in accordance with some embodiments of the disclosure provided herein. Table 800 comprises material column 810, PD₁ value column 820, PD₂ value column 830 and ratio column 840.

Material column 810 enumerates exemplary compositions disposed proximally to material characteristic signal detection device. It is by means exhaustive. PD₁ value column 820 represents intensities incident upon photodetector 650, in reference to FIG. 6. PD₂ value column 830 represents intensities incident upon photodetector 630, in reference to FIG. 6. Both columns are purposely labeled unitless, for these can represent a variety of different measure as described above.

In the present embodiment, the area of photodetector 630 (PD₂) is approximately twice that of photodetector 650 (PD₁) yield intensities of equal magnitude. The inventors of the present disclosure note that this increases accuracy in material identification; however, any geometry is not beyond the scope of the disclosure.

Ratio column 840 calculates and lists the ratio of PD₂/PD₁ for each material row. That is, PD₂ value over PD₁. Each row in table 800 gives rise to a characteristic signal for each material. In some embodiments, the ratio itself is used to exclude materials undesired materials. For example, in detecting the presence of finger tissue, ratios not near 1.46 would be excluded.

In other embodiments, PD₁ column value 820 and/or PD₂ column value 830 could be used to further develop a characteristic signal. In a further example, Teflon would not be readily excluded from finger tissue detection. However, the value of Teflon in PD₁ is roughly 4 times greater than that of finger tissue. Accordingly, Teflon could be excluded using the augmented characteristic signal or signature.

The inventors of the present disclosure exploit the notion that human skin has very specific characteristics compared to other scattering materials such as cloth etc. Furthermore, these characteristics are wavelength dependent. Thus, this ratio of PD₁/PD₂ is wavelength dependent and is also unique to skin.

In reference to the scattering theory of light, the inventors' observations and models closely conform, is consistent with and reflect this well-known theory, known to those in the art. As such, the observations that follow stem from an underlying physics and thus more general than any particular device enumerated in several of the embodiments.

That being said, the exact values of the ratios or the “characteristic signal” are dependent on the input light profile, position of the detectors and any other optics in the path. But, nevertheless, once the “geometry” is fixed, the ratios can be determined for any wavelength of operation (more wavelengths can be used to improve the test) by empirical measurements and thus “skin detection” can be carried out.

Other means, systems and devices are not beyond the scope of the present invention. For example, a plurality of 3 or more photodetectors could be used to further interpolate and extrapolate characteristic signals of materials. Additionally, alternate LED/photodetector dispositions and geometries can be used.

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein.

Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments may be implemented in any of numerous ways. One or more aspects and embodiments of the present application involving the performance of processes or methods may utilize program instructions executable by a device (e.g., a computer, a processor, or other device) to perform, or control performance of, the processes or methods.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above.

The computer readable medium or media may be transportable, such that the program or programs stored thereon may be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that may be employed to program a computer or other processor to implement various aspects as described above. Additionally, it should be appreciated that according to one aspect, one or more computer programs that when executed perform methods of the present application need not reside on a single computer or processor, but may be distributed in a modular fashion among a number of different computers or processors to implement various aspects of the present application.

Computer-executable instructions may be in many forms, such as program modules, executed by one or more computers or other devices. Generally, program modules include routines, programs, objects, components, data structures, etc. that performs particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a personal digital assistant (PDA), a smart phone, a mobile phone, an iPad, or any other suitable portable or fixed electronic device.

Also, a computer may have one or more input and output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

Elements other than those specifically identified by the “and/or” clause may optionally be present, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “between” is to be inclusive unless indicated otherwise. For example, “between A and B” includes A and B unless indicated otherwise.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

What is claimed is:

1. An apparatus for detecting a characteristic signal of a material comprising:
a light source;
a first photodetector disposed proximal to the light source;
a second photodetector disposed distally to the light source; and,
non-volatile logic for performing:
receiving a first signal from the first photodetector;
receiving a second signal from the second photodetector;
calculating a ratio of first and second signals; and,
determining the characteristic signal at least based on the ratio.

2. The apparatus of claim 1, wherein the light source comprises a first light emitting diode have a spectral intensity centered about a first wavelength, λ₁.

3. The apparatus of claim 2, wherein the light source further comprises a second light emitting diode have a spectral intensity centered about a second wavelength, λ₂.

4. The apparatus of claim 1, further comprising a substantially opaque barrier between the light source and first photodetector.

5. The apparatus of claim 1, further comprising a substrate.

6. The apparatus of claim 5, further comprising an analog front-end disposed on the substrate.

7. The apparatus of claim 6, wherein first photodetector and second photodetector are disposed on the analog front-end.

8. The apparatus of claim 5, wherein the light source is disposed on the substrate.

9. A method for identifying a characteristic signal of a material comprising:
emitting light from a light source through the material in close proximity to the light source;
receiving a first signal from a first photodetector disposed proximally to the light source;
receiving a second signal from a second photodetector disposed distally to the light source;
transmitting the first and second signal;
calculating a ratio of first and second signals; and,
determining the characteristic signal at least based on the ratio.

10. The method of claim 9, further comprising identifying the material as living tissue using the characteristic signal.

11. The method of claim 9, wherein the light source comprises a first light emitting diode have a spectral intensity centered about a first wavelength, λ₁.

12. The method of claim 11, wherein the light source comprises a second light emitting diode have a spectral intensity centered about a second wavelength, λ₂.

13. The method of claim 9, further comprising using a look-up table in determining a characteristic signal.

14. The method of claim 9, further comprising using one of the first or second photodetector signals and inverse ratio thereof in determining a characteristic signal.

15. The method of claim 9, wherein the area of the second photodetector is approximately twice that of the first photodetector.

16. The method of claim 9, wherein a substantial amount of the light received by the first and second photodetector passes through the material in close proximity.

17. The method of claim 9, further comprising transmitting light from the light source to the material.

18. The method of claim 17, further comprising receiving light from the material by the first and second photodetector.

19. The method of claim 18, wherein the received light has been scattered at least once within the material.

20. An apparatus for detecting the characteristic signal of a material comprising:
a means for transmitting light;
a first means for detecting light disposed proximal to the means for transmitting light;
a second means for detecting light disposed distally to the means for transmitting light;
a means for calculating a ratio of first and second signals; and,
a means for determining a characteristic signal of a material disposed in close proximity to the apparatus based on the ratio, at least in part.

21. The apparatus of claim 20, further comprising a means for looking up values in a look-up table in determination of the characteristic signal.

22. The apparatus of claim 20, further comprising a means for identifying the material.
